(19) United States
(12) Patent Application Publication
(10) Pub. No.: US 2017/0314008 A1
(43) Pub. Date: Nov. 2, 2017

(54) ENZYME IMMOBILIZATION USING IRON OXIDE YOLK-SHELL NANOSTRUCTURE

(71) Applicant: KONKUK UNIVERSITY INDUSTRIAL COOPERATION CORP., Seoul (KR)

(72) Inventors: Jung-Kul Lee, Seoul (KR); Yun-Chan Kang, Seoul (KR); Kumar singh sanjay Patel, Seoul (KR); Tae-Su Kim, Seoul (KR); Seung-Ho Choi, Gyeonggi-do (KR)

(21) Appl. No.: 15/507,487

(22) PCT Filed: Aug. 29, 2014
(86) PCT No.: PCT/KR2014/008085

§ 371 Date: Jul. 24, 2017

(51) Int. Cl. C12N 9/96; C12N 9/02; B01J 23/745; B01J 37/02; B01J 35/10; B01J 31/00; C02F 3/34; C02F 101/30

(52) U.S. Cl. CPC C12N 9/96; B01J 31/003; C12N 9/0061; C12Y 110/03002; C02F 3/342; B01J 23/745; B01J 37/0209; B01J 35/1061; C02F 2101/308; B01J 2231/64

(57) ABSTRACT

This invention relates to a carrier for immobilizing a biocatalyst including a Fe₂O₃ yolk-shell structure, to an immobilized enzyme using the carrier, and to realizing an increase in the stability of the enzyme and stability in organic solvents by cross-linking the enzyme. According to this invention, the carrier for immobilizing a biocatalyst and the enzyme immobilized thereon can be reused, have increased stability, facilitate the control of reactivity, pH, and temperature, and can be widely useful in various biochemical engineering industries.

### TECHNICAL FIELD

The present invention relates to a carrier for immobilizing an enzyme using a Fe₂O₃ yolk-shell structure, an immobilized enzyme using the carrier, a method of preparing the immobilized enzyme and the use thereof.

### BACKGROUND ART

The main purpose of enzyme immobilization is to make it easy to recover and reuse an enzyme, thus increasing the profitability of reaction processes using enzymes and enabling such reactions to be variously carried out in a batch manner or a continuous manner. Therefore, in order to effectively use a natural enzyme during a biochemical process, an enzyme has to be immobilized, and a typical enzyme immobilization process may include a physical adsorption process or a chemical process. A physical adsorption process is mainly performed through ion exchange, and an ion exchange process is advantageously non-toxic but is weak in bonding force. On the other hand, a chemical process is implemented using a chemical reagent so as to immobilize an enzyme by forming a covalent bond through a chemical reaction, and may exhibit a strong cross-linking force, but its use is limited in the food and pharmaceutical industries due to the toxicity of the reagent used to immobilize the enzyme.

As well known in the art, an enzyme immobilization process is performed in a manner in which an organic or inorganic carrier is coupled with an enzyme to immobilize the enzyme so as to conduct reuse and continuous treatment. The reason why an organic material (e.g. cellulose, nylon, polyacrylamide) is disadvantageous when used as a carrier is that the bonding thereof to the enzyme may break down due to poor mechanical stability, corrosion by the solvent, changes depending on pH and ionic intensity, and microbial infestation. Hence, an inorganic carrier to which an enzyme is adsorbed or covalently bonded is proposed, and the bonding type thereof may be dependent on the use conditions and morphologies of enzymes and the properties of substrates. Specifically, when a substrate has a strong salt concentration, the adsorbed enzyme may be detached, making it impossible to apply an adsorption process, and the covalent bonding of an enzyme thus takes precedence. The surface of the carrier has to possess a specific functional group that is able to induce the bonding of an enzyme. Since most carriers do not possess such functional groups, surface pretreatment thereof is required. The immobilization process via covalent bonding is performed in a manner in which the surface of the carrier and the enzyme are covalently bonded using a bonding agent or via a bridge, thus treating the surface of the carrier or introducing the functional group to the enzyme. Furthermore, the active site of the supported enzyme should not be blocked.

### CITATION LIST

Korean Patent Application Publication No. 1019880007719

### DISCLOSURE

### Technical Problem

The present invention has been made keeping in mind the problems encountered in the related art, and the present invention is intended to provide a novel carrier for enzyme immobilization.

In addition, the present invention is intended to provide a method of effectively immobilizing an enzyme.

### Technical Solution

Therefore, the present invention provides a carrier composition for immobilizing a biocatalyst, including a Fe₂O₃ yolk-shell structure.

In an embodiment of the present invention, the Fe₂O₃ yolk-shell structure preferably has one or more pores having an average diameter of 10 to 50 nm on the surface thereof.

As used herein, the term “surface” refers to a concept including not only the outermost shell surface but also one or more overlapping inner shell surfaces therein.

In addition, the present invention provides a method of immobilizing an enzyme using the carrier composition of the invention.

In an embodiment of the present invention, the method of immobilizing the enzyme preferably includes, but is not limited to, immobilizing an enzyme on the Fe₂O₃ yolk-shell structure and cross-linking the immobilized enzyme to form a crosslink.

In another embodiment of the present invention, the enzyme preferably includes, but is not limited to, a laccase enzyme.

In still another embodiment of the present invention, the cross-linking is preferably performed using glutaraldehyde, but is not limited thereto.

In addition, the present invention provides a Fe₂O₃ yolk-shell structure-enzyme complex composition, including a Fe₂O₃ yolk-shell structure on which an enzyme is immobilized.

In addition, the present invention provides a method of decolorizing a dye, including treating dye wastewater with the Fe₂O₃ yolk-shell structure-enzyme complex composition of the invention.

Hereinafter, a description will be given of the present invention.

In the present invention, in order to efficiently decolorize a dye from colored wastewater, a laccase enzyme is immobilized, and a commercial laccase enzyme is attached to a carrier activated by glutaraldehyde. In the present invention, the immobilization of the enzyme for decolorizing the dye from the colored wastewater creates the environment for long-term maintenance of the enzyme activity.

Adopted as the carrier for use in the enzyme immobilization according to the present invention is a Fe₂O₃ yolk-shell structure. The Fe₂O₃ yolk-shell structure is configured to have a predetermined sphere in which a movable small sphere is included and thus may exhibit superior absorptivity and may function as a porous carrier having adsorption capability for various kinds of proteins.

Under the above conditions, the enzyme immobilized on the Fe₂O₃ yolk-shell structure of the present invention is cross-linked, whereby the activity of the enzyme is maintained for a long time, and high stability of the enzyme and resistance thereof to organic solvents are ensured.

The laccase enzyme is immobilized on the optimal Fe₂O₃ yolk-shell structure and cross-linked, thus simultaneously ensuring stability and activity of the enzyme and resistance thereof to organic solvents, thereby maximizing productivity while significantly reducing production costs.

The carrier, which is configured such that the enzyme is immobilized on the Fe₂O₃ yolk-shell structure having a crosslink formed through cross-linking as described above, is useful in decolorization of dye from colored wastewater.

### Advantageous Effects

The present invention pertains to a carrier for immobilizing a biocatalyst including a Fe₂O₃ yolk-shell structure, to an immobilized enzyme using the carrier, to realizing an increase in the stability of the enzyme and stability in organic solvents by cross-linking the enzyme and to the use thereof. According to the present invention, the carrier for immobilizing a biocatalyst and the enzyme immobilized thereon can be reused, have increased stability, facilitate the control of reactivity, pH and temperature, and can be widely utilized in the food and pharmaceutical industries.

### BRIEF DESCRIPTION OF DRAWINGS

FIGS. 1A and B show electron microscope images of the surface of a Fe₂O₃ yolk-shell structure before and after immobilization of laccase on the Fe₂O₃ yolk-shell structure, and FIG. 1C shows an electron microscope image of the surface of the Fe₂O₃ yolk-shell structure;

FIG. 2 is a graph showing the FTIR absorbance when cross-linking laccase immobilized on the Fe₂O₃ yolk-shell structure;

FIG. 3 is a graph showing the optimal reaction temperature of laccase immobilized and cross-linked by the Fe₂O₃ yolk-shell structure, wherein: =pure laccase enzyme, ◯=laccase enzyme immobilized on the Fe₂O₃ yolk-shell structure, and ▾=laccase enzyme immobilized and then cross-linked on the Fe₂O₃ yolk-shell structure;

FIG. 4 is a graph showing the optimal reaction pH of laccase immobilized and cross-linked by the Fe₂O₃ yolk-shell structure, wherein: =pure laccase enzyme, ◯=laccase enzyme immobilized on the Fe₂O₃ yolk-shell structure, and ▾=laccase enzyme immobilized and then cross-linked on the Fe₂O₃ yolk-shell structure;

FIG. 5 is a graph showing the stability of the enzyme depending on the number of cycles of reuse of laccase immobilized on the Fe₂O₃ yolk-shell structure, wherein: grey square=laccase enzyme immobilized on the Fe₂O₃ yolk-shell structure and ▪=laccase enzyme immobilized and then cross-linked on the Fe₂O₃ yolk-shell structure; and

FIG. 6 is a graph showing the stability of the enzyme depending on the number of cycles of reuse of laccase immobilized on the Fe₂O₃ yolk-shell structure, regarding resistance of the cross-linked immobilized enzyme to the organic solvent, wherein: ▪=pure laccase enzyme and grey square=laccase enzyme immobilized and then cross-linked on the Fe₂O₃ yolk-shell structure.

### MODE FOR INVENTION

A better understanding of the present invention may be obtained via the following non-limiting examples, which are set forth to illustrate, but are not to be construed as limiting the scope of the present invention.

### Example 1: Synthesis of Fe₂O₃ Yolk-Shell Structure Using Spray Pyrolysis

The corresponding Fe₂O₃ yolk-shell structure was synthesized using a spray pyrolysis process as follows. A metal salt and dextrin as a drying aid are dissolved to give a transparent spray solution, which is then dried using a spray drying process, thereby synthesizing a metal oxide-carbon complex powder. The metal oxide-carbon complex is mass produced and then subjected to simple post-heat treatment at 300° C. or more, thus synthesizing a yolk-shell structure through stepwise combustion of the carbon complex. The detailed synthesis conditions are described below.
Preparation of solution: 0.15 M Fe nitrate is added to distilled water and completely dissolved. 10 g of dextrin is dissolved in 200 ml of an aqueous solution.
The prepared solution is sprayed into a spray-drying reactor using a nozzle, thus recovering particles.
Preparation conditions (spray-drying device operating conditions): an inlet temperature of 300° C., an outlet temperature of 120° C., and a nozzle pressure of 2.4 bar.
Reagents: iron nitrate (Junsei), dextrin (Samchun)

Using a transmission electron microscope, the Fe₂O₃ yolk-shell structure was observed before and after immobilization with laccase (FIG. 1: A-before immobilization, B-after immobilization). As shown in C of FIG. 1, the Fe₂O₃ yolk-shell structure is configured to have a predetermined sphere in which a movable small sphere is included, with porous particles having a size of 21 nm. Based on the results of analysis with a transmission electron microscope, multiple shells of the Fe₂O₃ yolk-shell structure are produced due to the stepwise combustion of dextrin. Conventional micrometer-sized particles are able to immobilize an enzyme only on the outermost portion thereof, whereas the yolk-shell Fe₂O₃ structure enables the immobilization of the enzyme up to the inside of the particles, thus making it possible to immobilize an enzyme in a large amount per unit volume and mass, namely in an amount at least three to four times the amount of conventional micrometer-sized particles. In the present invention, as the enzyme support, a Fe₂O₃ yolk-shell structure having superior performance was synthesized.

### Example 2: Immobilization of Laccase Enzyme

The Fe₂O₃ yolk-shell nanostructure is activated through treatment with glutaraldehyde as follows. Specifically, the Fe₂O₃ yolk-shell nanostructure is washed two times with distilled water. Thereafter, the Fe₂O₃ yolk-shell nanostructure is treated with 1 M glutaraldehyde. Then, in order to aid activation, reaction is carried out in a shaking incubator at 25° C. and 250 rpm for 4 hr. The activated Fe₂O₃ yolk-shell nanostructure is washed with 30 ml of distilled water and then washed once with a 100 mM phosphate buffer (pH 7).

10 mg of the activated carrier and 1 mg of a purified enzyme are mixed with a 50 mM phosphate buffer (pH 7) and then reacted in a shaking incubator at 4° C. and 150 rpm for 24 hr. The protein not coupled with the activated carrier is washed with distilled water and a 100 mM phosphate buffer (pH 7).

### Example 3: Cross-Linking of Enzyme Immobilized on Fe₂O₃ Yolk-Shell Nanostructure

Cross-linking was performed to maximize the stability of immobilized laccase. The enzyme immobilized on the Fe₂O₃ yolk-shell nanostructure was treated with glutaraldehyde in various concentrations ranging from 0.01 to 1.00 M in the presence of a phosphate buffer at pH 7.0 (50 mM) under conditions of 4° C. 150 rpm and 2 to 8 hr.

### Example 4: Results of Cross-Linking of Laccase Immobilized on Fe₂O₃ Yolk-Shell Structure

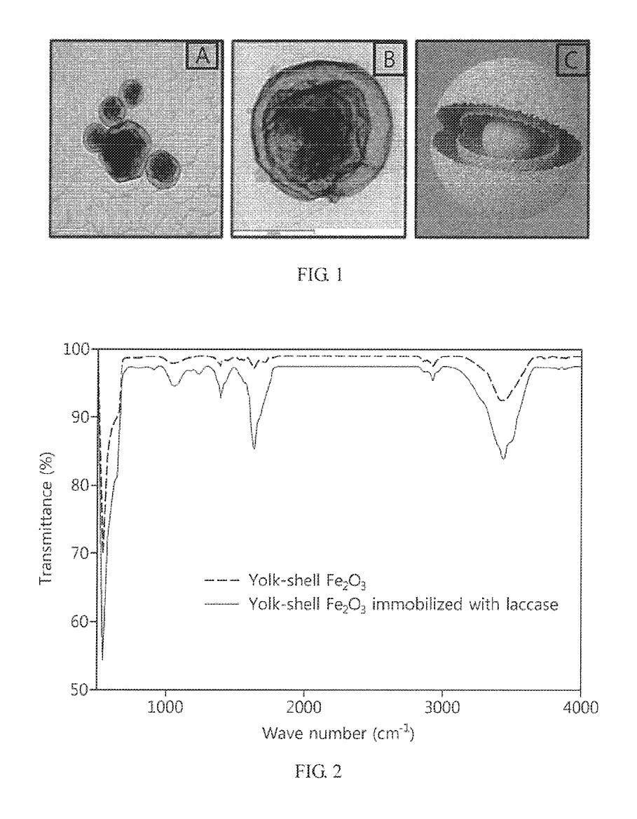

FIG. 2 is a graph showing the FTIR absorbance when cross-linking laccase immobilized on the Fe₂O₃ yolk-shell structure. As is apparent from the absorbance of 1600 to 1800 cm⁻¹ in the FTIR spectrum of FIG. 2, an amide bond (N═C═O) can be found to be formed due to the cross-linking.

### Example 5: Immobilization Efficiency of Laccase Enzyme Immobilized on Various Nano-Carriers

Using 2,2′-azino-bis(3-ethylbenzothiazoline-6-sulfonic acid) (ABTS, available from Sigma-Aldrich) as a substrate, the immobilized enzyme prepared as above and the enzyme immobilized on various carriers were measured for activity (Table 1). 1 mM ABTS and 0.05 μg of the immobilized enzyme were added to 1 ml of a reaction medium (50 mM sodium citrate buffer, pH 3.0), after which the oxidation of ABTS was carried out at a reaction temperature of 25° C. for 5 min. After the completion of the reaction for 5 min, the immobilized enzyme was separated from the reaction mixture using a magnet, and the product obtained through the oxidation of ABTS was analyzed by observing the absorbance at 420 nm.

Laccase was immobilized on each of commercial carriers and synthesized carriers, after which the immobilization yield (IY) and immobilization efficiency (IE) thereof were compared, whereby the immobilization yield was determined to range from 18.7 to 90.6% and the immobilization efficiency was determined to range from 18.4 to 87.5%. Under similar conditions among various carriers, the Fe₂O₃ yolk-shell structure exhibited the greatest immobilization yield of 90.6% and immobilization efficiency of 87.5%.

TABLE 1

| | | |
|---|---|---|
| | | Immobilization |
| Nano-particles | Immobilization yield (IY) % | Efficiency (IE) % |

Commercial particles

| | | |
|---|---|---|
| Al₂O₃ | 45.5 ± 3.7 | 37.8 ± 3.5 |
| SnO₂ | 18.7 ± 1.5 | 24.5 ± 2.1 |
| Fe₂O₃ | 64.2 ± 5.1 | 30.8 ± 2.6 |
| Fe₃O₄ | 37.4 ± 3.2 | 55.6 ± 5.1 |
| SiO₂ (15 nm) | 35.6 ± 3.0 | 48.4 ± 4.1 |
| SiO₂ (20 nm) | 48.2 ± 4.2 | 34.8 ± 3.0 |
| SiO₂ (80 mn) | 63.5 ± 5.3 | 69.0 ± 6.1 |
| SrFe₁₂O₁₉ | 42.5 ± 3.6 | 30.5 ± 2.5 |
| TiO₂ | 53.0 ± 4.1 | 40.1 ± 3.2 |
| Y₃Fe₅O₁₂ | 45.7 ± 3.8 | 23.2 ± 2.0 |
| ZrO₂ | 26.4 ± 2.1 | 18.4 ± 1.4 |

Synthesized particles

| | | |
|---|---|---|
| Fe₂O₃ yolk-shell | 90.6 ± 6.5 | 87.5 ± 7.1 |
| Fe₂O₃anti-cave | 44.5 ± 4.8 | 58.2 ± 4.6 |
| NiO@void@SiO₂ | 47.5 ± 4.2 | 52.1 ± 4.4 |
| Co₃O₄ (nanotube) | 42.4 ± 4.0 | 46.1 ± 4.0 |
| SnO₂ (Tube-in-Tube) | 48.6 ± 4.1 | 48.2 ± 4.2 |
| NiO@void@SiO₂ 10% | 53.8 ± 4.0 | 64.5 ± 5.1 |
| NiO@void@SiO₂ 40% | 59.1 ± 4.3 | 48.5 ± 3.8 |

Table 1 shows the immobilization efficiency of laccase on various nano-carriers.

### Example 6: Properties of Laccase Immobilized on Fe₂O₃ Yolk-Shell Structure Depending on Changes in Temperature

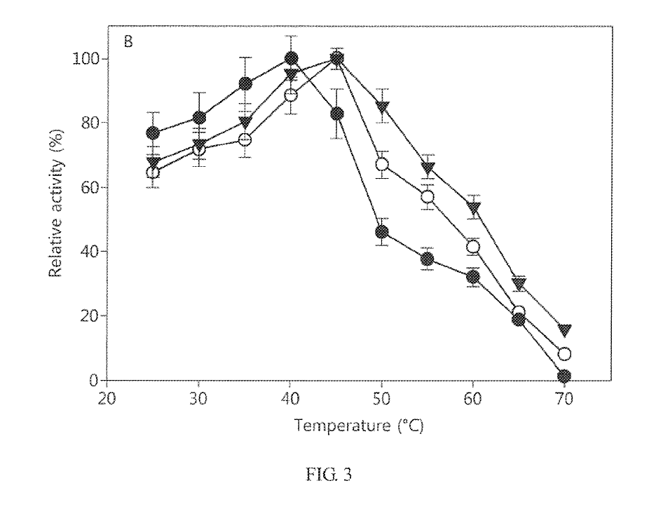

FIG. 3 shows the optimal temperatures of pure laccase, laccase immobilized on the Fe₂O₃ yolk-shell structure (YS-IM) and laccase obtained by cross-linking the immobilized enzyme (YS-IMC). Measurement was performed in the temperature range from 25 to 70° C. The optimal temperatures of the YS-IM and YS-IMC enzymes were 5° C. higher than that of the free laccase enzyme (FLac). Also, in the temperature range from 50 to 70° C., YS-IMC exhibited residual activity higher than those of FLac and YS-IM.

### Example 7: Properties of Laccase Immobilized on Fe₂O₃ Yolk-Shell Structure Depending on Changes in pH

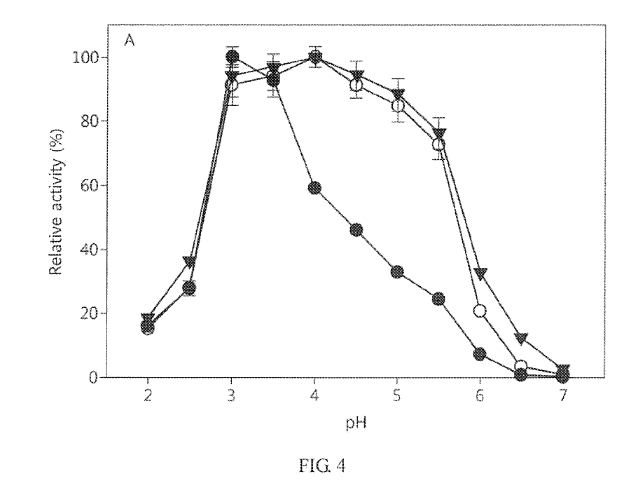

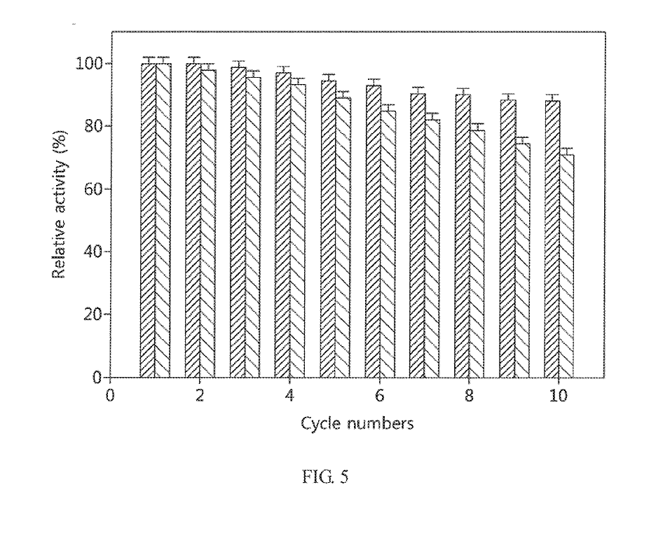

FIG. 4 shows the residual activity of laccase depending on changes in pH. The optimal pH was 3 for FLac, 4 for YS-IM, and 4 for YS-IMC. In the pH range of 5 to 7, the residual activity of YS-IMC was higher than those of FLac and YS-IM. That is, residual activity of YS-IMC was increased 2.7-, 4.5-, and 8.3-fold under the same conditions compared to FLac.

### Example 8: Stability of Laccase Upon Reaction Using Immobilized Enzyme

Changes in relative activity depending on the number of cycles of reuse of the immobilized enzyme were measured to determine the stability of the enzyme. The reaction was carried out at 25° C. using 1 mM ABTS and 0.05 μg of the immobilized enzyme. As shown in FIG. 5, ▪ and the grey square show changes in the relative activity depending on the number of cycles of reuse of YS-IMC and YS-IM, respectively. In FIG. 5, when the number of cycles of reuse reached 5 and 10, the relative activity of YS-IMC was 94.1 and 87.5% or more, and the relative activity of YS-IM was 88.6 and about 70.6%. Thus, the enzyme immobilized on YS-IMC was determined to be more stable.

### Example 9: Stability of Immobilized Laccase in Organic Solvent

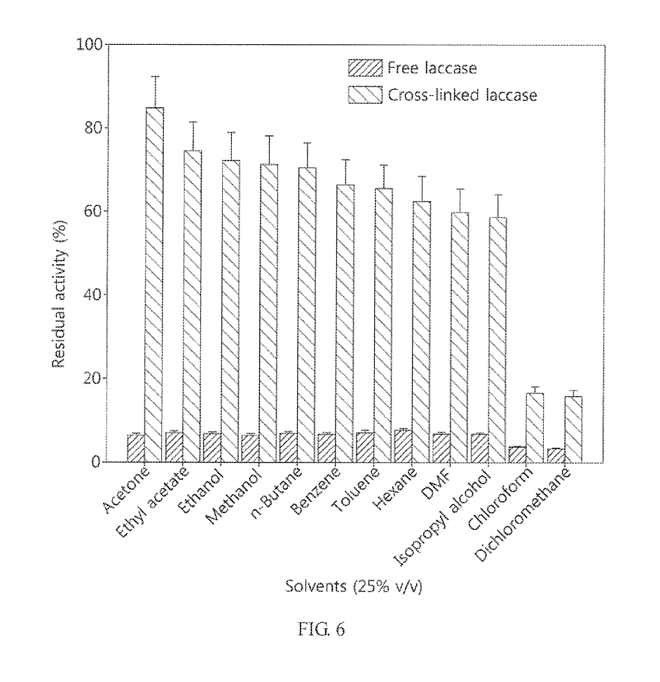

The resistance of FLac to 12 organic solvents (25% v/v) was evaluated through reaction at 25° C. for 4 hr. YS-IMC exhibited the residual activity of 15.8 to 84.7%, whereas the residual activity of FLac was only 8%. The organic solvent having the lowest toxicity to YS-IMC was acetone, and upon reaction for 4 hr and 12 hr, the residual activity was increased 13-fold and 32-fold respectively compared to FLac (FIG. 6).

1. A carrier composition for immobilizing a biocatalyst, comprising a Fe₂O₃ yolk-shell structure.

2. The carrier composition of claim 1, wherein the Fe₂O₃ yolk-shell structure has one or more pores having an average diameter of 10 to 50 nm on a surface thereof.

3. A method of immobilizing an enzyme using the carrier composition of claim 1.

4. The method of claim 3, comprising immobilizing an enzyme on the Fe₂O₃ yolk-shell structure and cross-linking the immobilized enzyme to form a crosslink.

5. The method of claim 3, wherein the enzyme is a laccase enzyme.

6. The method of claim 4, wherein the cross-linking is performed using glutaraldehyde.

7. A Fe₂O₃ yolk-shell structure-enzyme complex composition comprising a Fe₂O₃ yolk-shell structure on which an enzyme is immobilized.

8. The Fe₂O₃ yolk-shell structure-enzyme complex composition of claim 7, wherein the enzyme is a laccase enzyme.

9. A method of decolorizing a dye, comprising treating a dye wastewater with the Fe₂O₃ yolk-shell structure-enzyme complex composition of claim 7.
